(19) United States
(12) Patent Application Publication
(10) Pub. No.: US 2023/0158262 A1
(43) Pub. Date: May 25, 2023

(54) ENDOTRACHEAL TUBE

(71) Applicants: Konstantin Anatolievich PERVAK, Saint-Petersburg (RU); OBSCHESTVO S OGRANICHENNOYO OTVETSTVENNOSTYU "VLADIMED", Vladivostok (RU)

(72) Inventor: Konstantin Anatolievich PERVAK, Saint-Petersburg (RU)

(21) Appl. No.: 17/921,458

(22) PCT Filed: Apr. 28, 2021
(86) PCT No.: PCT/RU2021/050115

§ 371 Date: Oct. 26, 2022

(30) Foreign Application Priority Data
Apr. 29, 2020 (RU) 2020115099

(51) Int. Cl. A61M 16/04; A61M 19/00; A61M 16/08

(52) U.S. Cl. CPC A61M 16/044; A61M 16/0443; A61M 19/00; A61M 16/0486; A61M 16/0816

(57) ABSTRACT

An endotracheal tube (1) comprises a tubular body (2) having a form of a flexible curved transparent tube with open proximal (3) and distal (4) ends, a connector (7) mounted on the proximal end of the tube and served for attaching a tube cavity to a ventilation device, an inflatable cuff (9) having a cavity, said inflatable cuff fixed hermetically around the distal end of the curved tube and configured to be inflated to seal the cuff against a wall of a patient's trachea and to hold the distal end of the endotracheal tube in the desired position in a patient's larynx, a longitudinal air supply channel (10) in the wall of the tubular body, said channel being in communication with the cavity of the inflatable cuff, a sealed balloon (12) having elastic walls and configured to maintain and monitor pressure in the cuff cavity, the sealed balloon connected to an air supply port, and connected via a catheter (13) to the longitudinal air supply channel to supply an air into the cuff cavity, and a mark (14) made on the surface of the tube from the side of the proximal end at a distance of 2.5-3 cm from the inflatable cuff, wherein the inflatable cuff comprises two layers (15,16), wherein an outer layer (15) of the inflatable cuff is made of a porous biocompatible polymer material and serves as a depot for input and hold the drug in the pores of the polymer material and remove the drug from the pores of the zone of contacting of the cuff with the trachea when the cuff is inflated providing infusion of the drug into the tracheal mucosa.

### TECHNIAL FIELD

The present invention relates to medical technology, and more precisely to the endotracheal tube and can be used for surgical operations with anesthetic support, requiring intubation of the trachea with an endotracheal tube.

### BACKGROUND OF THE INVENTION

Endotracheal tubes are used in anesthesiology, intensive care, and critical care medicine to perform manipulations in the tracheobronchial tree and to administer various drugs.

Endotracheal tubes can also be used to protect and ensure the patency of the upper airways during transportation of wounded patients during medical evacuation.

In modern anesthesiology, there are serious problems concerning tracheal intubation. During intubation, such phenomena as reflex response associated with an increase in the tone of the nervus vagus, or stimulation of effector endings involved in reflex stimulation of the sympathetic nerves of the heart, may occur. All these phenomena cause hemodynamic responses that are dangerous for a patient's life.

It is well known that the larynx and trachea reflexes are strong, and at certain stages in order to interrupt the cough reflex and prevent reflex hemodynamic response, it is necessary to use deepen anesthesia.

In cases where long-term ventilation is necessary, laryngeal-pharyngeal reflexes make it difficult to synchronize the patient's breathing with the ventilator and worsen the quality of synchronization.

The most effective methods of reducing reflex response are methods of anesthesia of the larynx and trachea mucous with local anesthetics by using various sprays, atomizers and inhalers; or ointments containing local anesthetics. All these methods have significant drawbacks; in particular, the local anesthetic being administered with aerosols does not enter the subglottic space in the required amount, and to more distal parts of the respiratory tract. In the meantime, ointments with local anesthetics have a slow diffusion activity. However, the effective time of action of anesthetics is limited by the time of action of the local anesthetic, and there is a risk of both insufficient dosage and excess dosage.

Known from prior art is a double-wall surgical cuff for introduction into a body passage such as a trachea of a surgical fluid such as an anesthetic (see, for example, U.S. Pat. No. 4,417,576, published Nov. 29, 1983), the surgical cuff comprises a tubular base member with an imperforate inflatable tubular inner cuff member encircling the tubular base member and a distensible tubular outer cuff member encircling the inner cuff member. The outer cuff member is multiperforated at spaced points. A sponge-like material, like sponge rubber, is positioned in the space between the inner and outer cuff members. Separate passages communicate with the space occupied by the sponge-like material and the space between the tubular base member and the inner cuff member. Surgical fluid such as an anesthetic is introduced into the space with the sponge-like material, which absorbs it. Then air is pumped into the space between the tubular base member and the inner cuff member to expand the inner cuff member and, thus, compress the sponge-like material to drive the absorbed surgical fluid through the perforations in the wall of the outer cuff member to the adjacent walls of the body passage.

Disadvantages of the disclosed cuff include the risk of both insufficient dosage and excess dosage of the anesthetic removed from the sponge-like material. The cuff also does not ensure the delivery of the anesthetic in front of the distal end of the tube for preliminary anesthesia of the tracheal mucosa during intubation and uniform distribution of the anesthetic in the mucosal contact zone of the trachea with the cuff.

Known from prior art is an endotracheal tube incorporating a drag-irrigation device (see U.S. Pat. No. 5,146,916, published Sep. 15, 1992). The endotracheal tube (Catalani tube) (FIG. 1) is equipped for delivering a drug (anesthetic) externally of the tube in the zone of the trachea. The endotracheal tube comprises a tube body (2) having proximal end and distal end, and at least one flexible irrigation cannula extending along the endotracheal tube body to its distal end.

An irrigation diffuser means (12, 14) is attached to the irrigation cannula for spraying a drug delivered through the irrigation cannula externally of the endotracheal tube body. Said least one flexible irrigation cannula extending along said endotracheal tube body to said distal end is divided in its proximal portion into two branch cannulas; an irrigation diffuser means (12, 14) comprising a pair of chambers respectively circumscribed by a pair of cuffs located proximally and distally with respect to said balloon portion, respectively. Said cuffs each comprising a double wall, and outer wall of each cuff having a plurality of small holes for emitting a drug; and a pair of grooves opening outwardly of said tube body for respectively containing said pair of cuffs without substantial protrusion of said cuffs beyond a main outer surface of said body.

The endotracheal tube is particularly intended for artificial ventilation in surgical operations and in intensive resuscitation treatments. It is useful for the repeated administration and re-administration of drugs, for instance, of local anesthetics, anti-inflammatories and mucolytics in the course of intubation. The endotracheal tube allows delivery of a drug, anesthetic to the contact area of the cuff and the tracheal mucosa, but has a number of significant disadvantages. The presence of annular channels leads to the appearance of kinks of the tube at the locations of the annular channels, especially in the area of the upper annular channel at small diameters of the tube.

When using this tube, an unregulated amount of drug accumulates between the outer and inner cuff shells, and the presence of a large number of holes in the cuff leads to excessive drug delivery.

Endotracheal tubes according to prior art are of various shapes and designs and have secondary channels for various purposes, but none of the known endotracheal tubes is designed to effectively administer drugs such as local anesthetics in a given single dose, none of the tubes provide a decrease the dose of the drugs used in general anesthesia, none of the tubes ensures a decrease a risk of excess dosage, as well as the most uniform distribution of anesthetic in the zone of contact of the tracheal mucosa with the cuff of the endotracheal tube.

### SUMMARY OF INVENTION

Accordingly it is an object of the present invention to provide an endotracheal tube, comprising an inflatable cuff, that overcomes the shortcomings of conventional devises, the design of which ensures

delivery of a drug in the amount of a single dose, and, due to this, to reduce the dose of the drug used in general anesthesia and avoid a risk of excess dosage,

the most uniform distribution of the anesthetic in the zone of contact of the tracheal mucosa with the cuff of the endotracheal tube, which ensures a significant decrease in laryngeal-pharyngeal reflexes when applying anesthesia,

ensures delivery of drugs in front of the distal end of the tube for preliminary anesthesia of the tracheal mucosa during intubation, as well as increase the safety of anesthesia and its effectiveness,

and also to exclude the ingress of mucus and vomit into the bronchi.

According to the first aspect of the invention proposed is an endotracheal tube, the endotracheal tube comprising:

a tubular body having a form of a flexible curved transparent tube with open proximal and distal ends,

a connector mounted on the proximal end of the tube and served for attaching a tube cavity to a ventilation device,

an inflatable cuff having a cavity, said inflatable cuff fixed hermetically around the distal end of the curved tube and configured to be inflated to seal the cuff against a wall of a patient's trachea and to hold the distal end of the endotracheal tube in the desired position in a patient's larynx,

a longitudinal air supply channel in the wall of the tubular body, said channel being in communication with the cavity of the inflatable cuff,

a sealed balloon having elastic walls and configured to maintain and monitor pressure in the cuff cavity, the sealed balloon connected to an air supply port, and connected via a catheter to the longitudinal air supply channel to supply an air into the cuff cavity, and

a mark made on the surface of the tube from the side of the proximal end at a distance of 2.5-3 cm from the inflatable cuff,

according to the invention the inflatable cuff comprises two layers, wherein the outer layer of the inflatable cuff is made of a porous biocompatible polymer material and serves as a depot for input and hold the drug in the pores of the polymer material and remove the drug from the pores of the zone of contacting of the cuff with the trachea when the cuff is inflated providing infusion of the drug into the tracheal mucosa.

Preferably the outer porous layer of biocompatible polymeric material is thickened in the area located in the middle of the inflatable cuff, in the zone of contact of the cuff with the trachea.

Preferably the inner layer of the inflatable cuff is made of homogeneous polyvinyl chloride, ensuring to inflate the cuff to seal the cuff against a wall of a patient's trachea to hold it in the trachea.

Preferably the outer layer of the cuff is made of porous polyvinyl chloride.

Preferably a local anesthetic is used as the drug.

Preferably the drug is a form of a solution, suspension or ointment held in the pores of the porous material of the inflatable cuff.

Preferably a single dose drug is used as an amount of the drug held in the pores of the porous material of the outer layer.

Preferably the endotracheal tube comprises an additional mark located in the immediate vicinity of the first mark from the distal end of the endotracheal tube at a distance of about 0.5 cm from the first mark.

According to the second aspect of the invention proposed is an endotracheal tube, the endotracheal tube comprises:

a tubular body having a form of a flexible curved transparent tube with open proximal and distal ends, and having a convex and a concave sides,

a connector mounted on the proximal end of the tube and served for attaching a tube cavity to a ventilation device,

an inflatable cuff having a cavity, said inflatable cuff fixed hermetically around the distal end of the curved tube and configured to be inflated to seal the cuff against a wall of a patient's trachea and to hold the distal end of the endotracheal tube in the desired position in a patient's larynx,

a longitudinal air supply channel in the wall of the tubular body, said channel being in communication with the cavity of the inflatable cuff,

a sealed balloon having elastic walls and configured to maintain and monitor pressure in the cuff cavity, the sealed balloon connected to an air supply port, and connected via a catheter to the longitudinal air supply channel to supply an air into the cuff cavity,

a longitudinal drag supply channel, the channel made in the wall of the tubular body and connected by means of a catheter to a metered-dose drug supply container, wherein the central angle between the longitudinal air supply channel and the longitudinal drag supply channel is in the range of 90-180 degrees,

at least two marks made on the surface of the tube, the first mark is located in the immediate vicinity of the cuff, the second mark is located at a distance of about 2-2.5 cm from the first mark, closer to the proximal end of the tube, so that said second mark is seen from the laryngopharynx when the endotracheal tube is in the required working position of the endotracheal tube in the patient's larynx at the border of the upper part of the larynx,

two channels in the tubular body in a plane perpendicular to the axis of the tube, the channels opened to the outside of the tube in the immediate vicinity of the second mark from the side of the proximal end of the tube, said two channels communicating with the longitudinal drag supply channel and having each a cone-shaped expanding outlet,

two channels in the tubular body in a plane perpendicular to the axis of the tube, the channels opened to the outside of the tube in the immediate vicinity of the cuff between said first mark and the cuff, said two channels communicating with the longitudinal drag supply channel and having each a cone-shaped expanding outlet, and

two channels in the tubular body in a plane perpendicular to the axis of the tube, the channels opened to the outside of the tube in the immediate vicinity of the distal end of the tube between the cuff and the distal end of the tube, said two channels communicating with the longitudinal drag supply channel and having each a cone-shaped expanding outlet,

according to the invention the inflatable cuff comprises two layers, wherein the outer layer of the inflatable cuff is made of a porous biocompatible polymer material and serves as a depot for input and hold the drug in the pores of the polymer material and remove the drug from the pores of the zone of contacting of the cuff with the trachea when the cuff is inflated providing infusion of the drug into the tracheal mucosa.

Preferably, the outer porous layer of biocompatible polymeric material is thickened in the area located in the middle of the inflatable cuff, in the zone of contact of the cuff with the trachea.

Preferably the inner layer of the inflatable cuff is made of homogeneous polyvinyl chloride, ensuring to inflate the cuff to seal the cuff against a wall of a patient's trachea to hold it in the trachea.

Preferably the outer layer of the cuff is made of porous polyvinyl chloride.

Preferably a local anesthetic is used as the drug.

Preferably the drug is a form of a solution, suspension or ointment held in the pores of the porous material of the outer layer.

Preferably a single dose drug is used as an amount of the drug held in the pores of the porous material of the inflatable cuff.

Preferably the endotracheal tube comprises an additional mark located in close proximity to the second mark on the side of the first mark.

According to the third aspect of the invention proposed is an endotracheal tube, the endotracheal tube comprises:

a tubular body having a form of a flexible curved transparent tube with open proximal and distal ends, and having a convex and a concave sides,

a connector mounted on the proximal end of the tube and served for attaching a tube cavity to a ventilation device,

an inflatable cuff having a cavity, said inflatable cuff fixed hermetically around the distal end of the curved tube and configured to be inflated to seal the cuff against a wall of a patient's trachea and to hold the distal end of the endotracheal tube in the desired position in a patient's larynx,

a longitudinal air supply channel in the wall of the tubular body, said channel being in communication with the cavity of the inflatable cuff,

a sealed balloon having elastic walls and configured to maintain and monitor pressure in the cuff cavity, the sealed balloon connected to an air supply port, and connected via a catheter to the longitudinal air supply channel to supply an air into the cuff cavity,

a longitudinal drag supply channel, the channel made in the wall of the tubular body and connected by means of a catheter to a metered-dose drug supply container, wherein the central angle between the longitudinal air supply channel and the longitudinal drag supply channel is in the range of 90-180 degrees,

at least two marks made on the surface of the tube, the first mark is located in the immediate vicinity of the cuff, the second mark is located at a distance of about 2-2.5 cm from the first mark, closer to the proximal end of the tube, so that said second mark is seen from the laryngopharynx when the endotracheal tube is in the required working position of the endotracheal tube in the patient's larynx at the border of the upper part of the larynx,

two channels in the tubular body in a plane perpendicular to the axis of the tube, the channels opened to the outside of the tube in the immediate vicinity of the second mark from the side of the proximal end of the tube, said two channels communicating with the longitudinal drag supply channel and having each a cone-shaped expanding outlet,

an annular drag supply channel for supplying the drag to the area of contact of the cuff with the tracheal mucosa and to underlying parts of the respiratory tract, the annular drag supply channel made in the tube wall in the immediate vicinity of the inflatable cuff from the proximal end of the tube, between the cuff and the first mark, the annular drag supply channel communicating with the longitudinal drag supply channel and comprising:

at least three pairs of holes including the first pair, the second pair and the third pair, the holes having axes of different directions, the holes are located along the circumference at a distance from each other, wherein the first two pairs of holes having a cone-shaped expanding shape with a large cross-sectional diameter closer to the outer surface of the wall of the tube, the axis of each hole of the first pair is located at an angle of 45 degrees to the axis of the tube and is directed towards the distal end of the tube, the axis of each hole of the second pair is placed at an angle of 90 degrees to the axis of the tube, the third pair of holes has a cylindrical shape and the axes of the holes of the third pair are located at an angle of 25-30 degrees to the longitudinal axis of the tube and are directed towards the distal end of the tube,

according to the invention the inflatable cuff comprises two layers, wherein the outer layer of the inflatable cuff is made of a porous biocompatible polymer material and serves as a depot for input and hold the drug in the pores of the polymer material and remove the drug from the pores of the zone of contacting of the cuff with the trachea when the cuff is inflated providing infusion of the drug into the tracheal mucosa.

Preferably, the outer porous layer of biocompatible polymeric material is thickened in the area located in the middle of the inflatable cuff, in the zone of contact of the cuff with the trachea.

Preferably a single dose drug is used as an amount of the drug held in the pores of the porous material of the outer layer.

Preferably the inner layer of the inflatable cuff is made of homogeneous polyvinyl chloride, ensuring to inflate the cuff to seal the cuff against a wall of a patient's trachea to hold it in the trachea.

Preferably the outer layer of the cuff is made of porous polyvinyl chloride.

Preferably a local anesthetic is used as the drug.

Preferably the drug is a form of a solution, suspension or ointment held in the pores of the porous material of the inflatable cuff.

Preferably the endotracheal tube comprises an additional mark in close proximity to the second mark on the side of the first mark.

The technical effect provided by the claimed invention is that the design of the endotracheal tube comprising the inflatable cuff ensures local anesthesia in the area of contact of the cuff with the tracheal mucosa practically immediately after intubation due to the uniform release of local anesthetic from the porous layer of the cuff, delivery of a drug in the amount of a single dose, and due to this, to reduce the dose of the drug used in general anesthesia and avoid a risk of excess dosage.

After the release of the local anesthetic from the porous material of the cuff, the design of the tube provides the possibility of supplying drugs to the cuff area and the space under ligaments, ensures the delivery of drugs along the entire length of the tube in the areas of the greatest contact with the mucous of trachea, pharynx and larynx, and provides early drug delivery during intubation until the final installation of the tube in the trachea. Kinks of the tube in the zone of the annular channel, if any, are excluded, and the turbulence of the air flow inside the tube itself is reduced.

The proposed design of the endotracheal tube provides high-quality local anesthesia of the mucous of the oropharynx and trachea, delivery of a drug in the amount of a single dose, a significant reduction of the dosage of general drugs, namely anesthetics, analgesics and muscle relaxants, that is extremely important for patients in critical condition, the most uniform distribution of the anesthetic in the zone of contact of the tracheal mucosa with the cuff of the endotracheal tube, which ensures a significant decrease in laryngeal-pharyngeal reflexes when applying anesthesia, increase the safety of anesthesia and its effectiveness, and also exclude the ingress of mucus and vomit into the bronchi.

Presence of multi-vector holes in the annular channel allows:

irrigate the area of the cuff in contact with the tracheal mucosa by injecting drugs through the cone-shaped holes located at an angle of 40-45 degrees to the tube axis, through the cylindrical holes located at an angle of 20-25 degrees to the tube axis, allowing irrigation of the tracheal mucosa during intubation in front of the distal end of the endotracheal tube, and through the cone-shaped holes located at an angle of 90 degrees allowing irrigation directly to the contact area of the cuff with the mucous during manipulation of its downward movement and subsequent return of the tube.

Presence of three marks provides monitoring of the location of the endotracheal tube during drug delivery at all stages of the anesthesia, avoiding administering of drugs onto the glottis. The mark that is closed to the distal end allows to control the initial moment of drug administration, after said mark passes through the glottis during intubation, providing preliminary anesthesia of the tracheal mucosa before placing the endotracheal tube in the working position. An additional mark ensures the correct positioning of the lower marking in relation to the glottis. The second mark allows to perform anesthesia of the spaces over and under ligaments, avoiding the drug getting into the glottis.

These and other objects, features, and characteristics of the present invention, as well as the methods of operation and functions of the related elements of structure, will become more apparent upon consideration of the following description and the appended claims with reference to the accompanying drawings, all of which form a part of this specification, wherein like reference numerals designate corresponding parts in the various figures. It is to be understood, however, that the drawings are for the purpose of illustration and description only and are not intended as a definition of the limits of the invention.

### BRIEF DESCRIPTION OF THE DRAWINGS

FIG. 1 schematically illustrates the known Catalani tube (prior art);

FIG. 2 schematically illustrates a general view of the endotracheal tube (first embodiment), according to the invention;

FIG. 3 schematically illustrates an inflated cuff placed on the tube;

FIG. 4 schematically illustrates the endotracheal tube, the cuff is located near the distal end of the tube and two marks near the cuff from the side of the proximal end of the tube, according to the first embodiment;

FIG. 5 schematically illustrates an endotracheal tube, three marks near the cuff from the proximal end of the tube, and three transverse drug delivery channels in the body of the tube according to the second embodiment;

FIG. 6 schematically illustrates a section along the line VI-VI in FIG. 5, two channels in communication with the drug delivery channel;

FIG. 7 schematically illustrates a general view of the endotracheal tube (second embodiment), according to the invention;

FIG. 8 schematically illustrates an endotracheal tube, the cuff is placed near the distal end of the tube; three marks near the cuff from the proximal end of the tube; two transverse drug delivery channels in the body of the tube, an annular channel for drug delivery, according to the third embodiment, the directions of drug jets are schematically shown, the drug is supplied from the holes made in the annular channel;

FIG. 9 schematically illustrates a section along line IX-IX in FIG. 8;

FIG. 10 schematically illustrates a cone-shaped hole in an annular channel, the axis of the hole runs at an angle of 45 degrees to the tube axis;

FIG. 11 schematically illustrates a cone-shaped hole made in the annular channel, the axis of the hole runs at an angle of 90 degrees to the tube axis;

FIG. 12 schematically illustrates a cylindrical hole made in an annular channel, the axis of the hole runs at an angle of 25-30 degrees to the tube axis;

FIG. 13 schematically illustrates the cuff placed near the distal end of the tube, the outer porous layer of biocompatible polymeric material is thickened in the area located in the middle of the inflatable cuff, in the zone of contact of the cuff with the trachea.

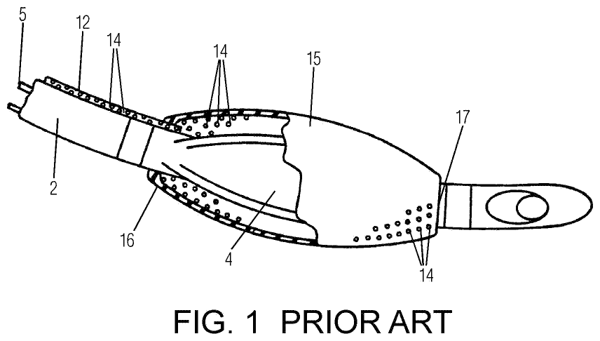

### DETAILED DESCRIPTION OF THE PREFERRED EMBODIMENTS

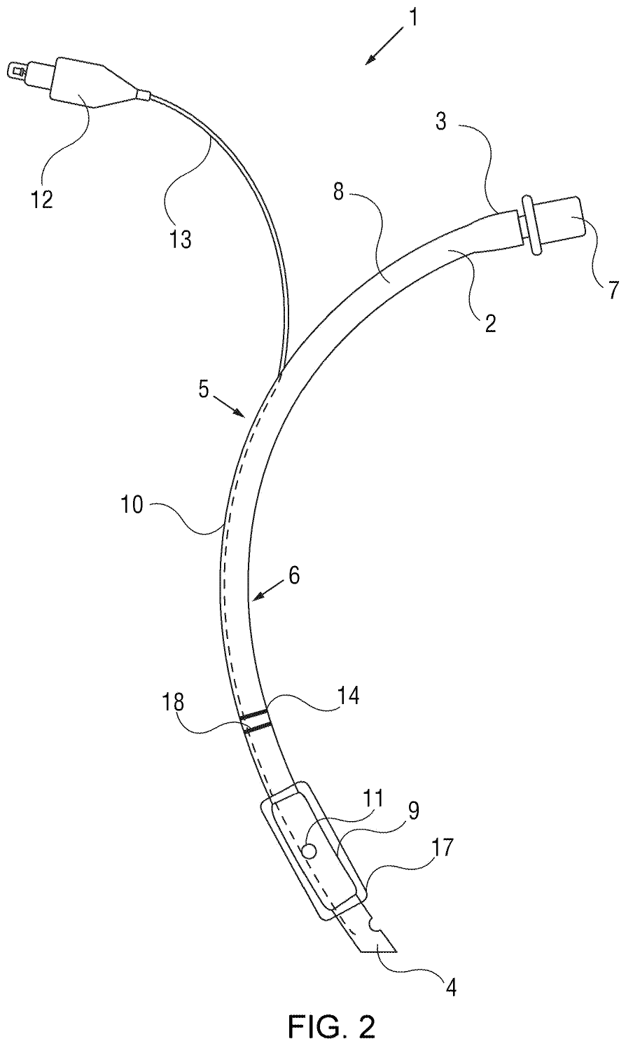

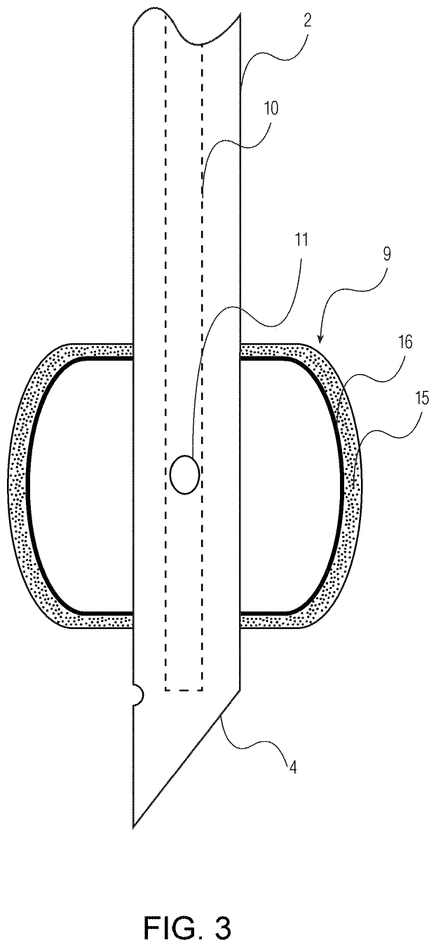

An endotracheal tube 1 (FIG. 2) according to the claimed invention, comprises a tubular body 2 having a form of a flexible curved transparent tube with open proximal 3 and open distal 4 ends, and having a convex 5 and a concave 6 sides.

According to the first embodiment, the endotracheal tube 1 comprises a connector 7 mounted on the proximal end 3 of the tube 2 and served for attaching a tube cavity 8 to a ventilation means (not shown), and an inflatable cuff 9 having a cavity. Said inflatable cuff 9 is fixed hermetically around the distal end 4 of the curved tube 2 and configured to be inflated to seal the cuff 9 against a wall of a patient's trachea and to hold the distal end 4 of the endotracheal tube in the desired position in a patient's larynx.

A longitudinal air supply channel 10 is made in the wall of the tubular body 2 and is in communication with the cavity of the inflatable cuff 9 through a hole 11 in the wall of the tube 2.

A sealed balloon 12 having elastic walls is configured to maintain and monitor pressure in the cuff cavity, the sealed balloon 12 is connected to an air supply port (not shown), and connected via a catheter 13 to the longitudinal air supply channel 10 to supply an air into the cuff cavity.

A mark 14 is made on the surface of the tube 2 from the side of the proximal end 3 at a distance of 2.5-3 cm from the inflatable cuff 9.

According to the invention, the inflatable cuff 9 comprises two layers 15 and 16, wherein the outer layer 15 of the inflatable cuff 9 is made of a porous biocompatible polymer material and serves as a depot for input and hold the drug in the pores of the polymer material and remove the drug from the pores in the zone of contacting of the cuff 9 with the trachea when the cuff 9 is inflated providing infusion of the drug into the tracheal mucosa. In the drawings, the inflatable cuff 9 is shown in an inflated state.

Preferably, the outer porous layer 15 of biocompatible polymeric material is thickened in the area located in the middle of the inflatable cuff 9, in the zone of contact of the cuff with the trachea.

The inner layer 16 of the inflatable cuff 9 is made of homogeneous polyvinyl chloride, ensuring to inflate the cuff to seal the cuff against a wall of a patient's trachea to hold it in the trachea. The outer layer 15 of the cuff 9 is made of porous polyvinyl chloride.

The drug is supplied to the trachea zone for anesthesia as a result of drug infusion from the porous outer layer 15 of the cuff during its inflation and diffusion of the drug, the drug will be delivered when the porous outer layer 15 of the cuff 9 contacts the tracheal mucosa due to the high adsorption capacity of the tracheal mucosa. The configuration of the cuff 9 allows the drug to be applied and distributed evenly over the entire surface of the cuff, which is carried out in the factory.

The drug is a form of a solution, suspension or ointment held in the pores of the porous material of the inflatable cuff 9.

After applying the drug, the cuff 9 is packed in a sealed package 17 (FIG. 2), the package is sterile, impermeable and tight to the cuff 9.

A local anesthetic is used as a drug injected into the pores of the cuff 9.

The introduction of the drug into the pores of the outer layer 15 of the cuff 9 is carried out at the factory immediately after the manufacture of the endotracheal tube. The amount of drug applied and held in the pores of the porous material of the outer layer 15 of the inflatable cuff 9 is a single dose of local anesthetic required for initial anesthesia. After packing the cuff 9 into a sterile package 17, the endotracheal tube 1 is placed in a sterile bag and transferred to storage. It helps to delivery of a drug in the amount of a single dose, and due to this, to reduce the dose of the drug used in general anesthesia and avoid a risk of excess dosage.

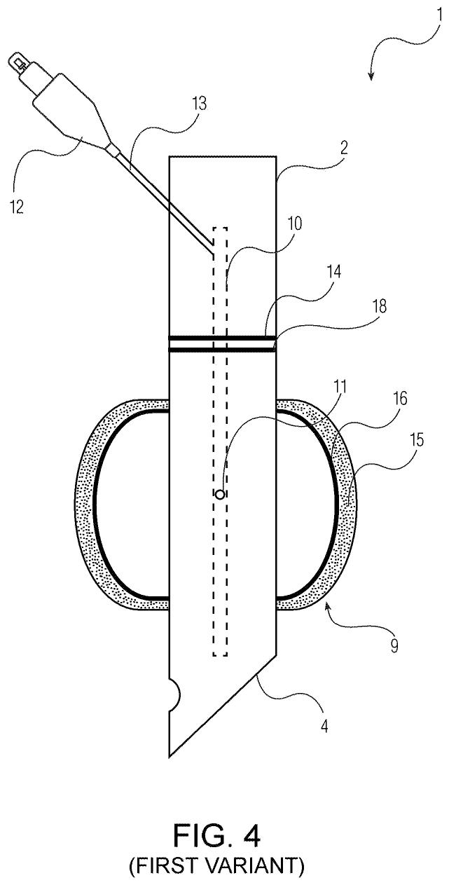

In this embodiment the endotracheal tube comprises an additional mark 18 (FIG. 4) located in the immediate vicinity of the first mark 14 from the distal end 4 of the endotracheal tube at a distance of about 0.5 cm from the first mark 14.

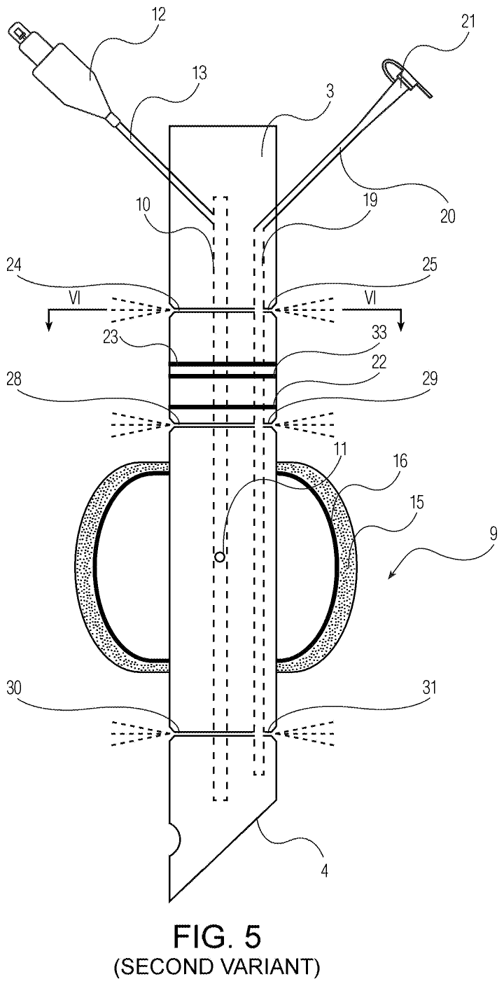

According to the second embodiment, the endotracheal tube 1 (FIG. 5), in addition to the first embodiment, comprises a longitudinal drug supply channel 19 made in the wall of the tube body 2 and connected by means of a catheter 20 to a metered-dose drug supply container 21. The central angle between the longitudinal air supply channel 10 and the longitudinal drug supply channel 19 is in the range of 90-180 degrees. The air supply channel 10 is connected to the cuff cavity through the opening 11.

At least two marks 22, 23 (FIG. 5) are made on the surface of the tube body 2, the first mark 22 is located in the immediate vicinity of the cuff 9, the second mark 23 is located at a distance of about 2-2.5 cm from the first mark 22, closer to the proximal end 3 of the tube. The position of the second mark 23 is such that said second mark 23 is seen from the laryngopharynx when the endotracheal tube is in the required working position in the patient's larynx at the border of the upper part of the larynx. Said marks 22 and 23 are similar to marks 14 and 18 of the first embodiment of the endotracheal tube.

The working position of the tube is the position at which the distal end of the tube at the ending of intubation is in a position that is from 2 to 4 cm above the tracheal bifurcation. In this case, the glottis is located between the upper mark, i.e. the mark that is located closest to the proximal end of the tube, and the intermediate mark.

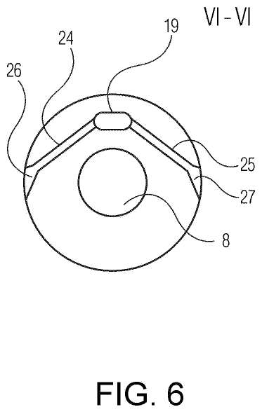

The endotracheal tube comprises two channels 24, 25 (FIG. 5) in the tubular body 2 in a plane perpendicular to the axis of the tube, the channels 24, 25 are opened to the outside of the tube in the immediate vicinity of the second mark 23 from the side of the proximal end 3 of the tube. Said two channels 24, 25 (FIG. 6) are located at an angle to each other and communicate with the longitudinal channel 19 for supplying drugs and each channel has cone-shaped expanding outlet 26, 27 of a circular or oval shape.

The endotracheal tube comprises two channels 28, 29 (FIG. 5) that are similar to channels 24, 25; channels 28, 29 are made in the tubular body 2 in a plane perpendicular to the axis of the tube and come out to the surface of the tube in the immediate vicinity of the cuff 9 between the first mark 22 and the cuff. Channels 28, 29 communicate with a longitudinal drug delivery channel 19 and each channel has cone-shaped expanding outlet.

The endotracheal tube comprises two more channels 30, 31 (FIG. 5) that are similar to channels 24, 25; channels 30, 31 are made in the tubular body 2 in a plane perpendicular to the axis of the tube and come out to the surface of the tube in the immediate vicinity of the distal end 4 of the tube between the cuff 9 and the distal end 4 of the tube. Channels 30, 31 communicate with a longitudinal drug delivery channel 19 and each channel has cone-shaped expanding outlet.

In the described second embodiment, the inflatable cuff 32 is similar to the cuff 9 of the first embodiment of the endotracheal tube.

The endotracheal tube 1 in the described second embodiment comprises an additional mark 33 located in the immediate vicinity of the second mark 23 from the side of the first mark 22, at a distance of about 0.5 cm from the mark 23.

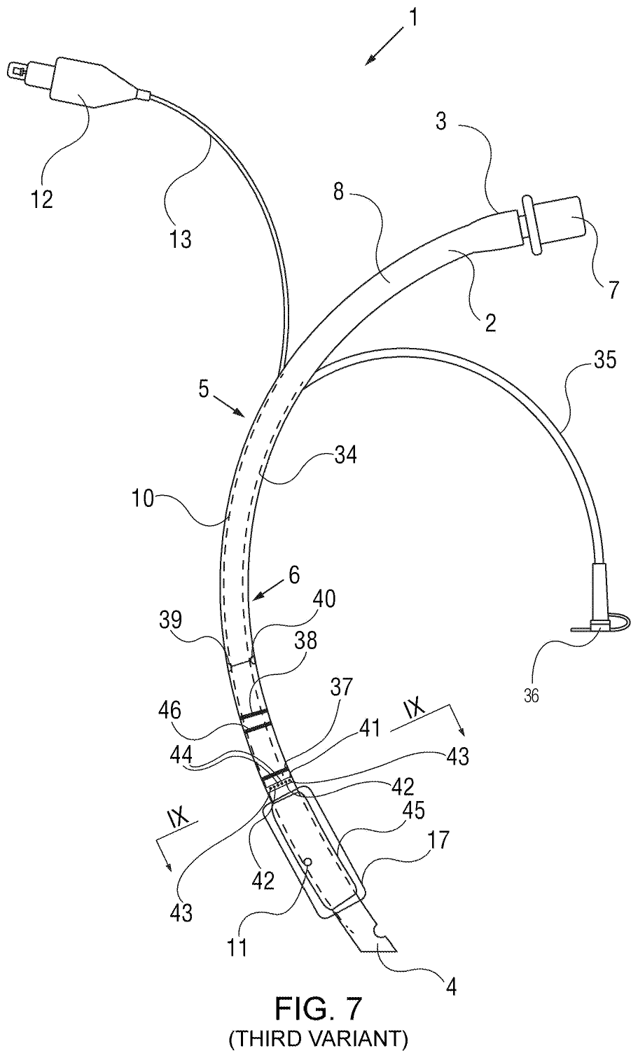

According to the third embodiment, the endotracheal tube 1 (FIG. 7), in addition to the first embodiment, comprises a longitudinal drug supply channel 34 made in the wall of the tube body 2 and connected by means of a catheter 35 to a container 36 for dispensing drugs. The central angle between the air supply channel 10 and the drug supply channel 34 is in the range of 90-180 degrees. The air supply channel 10 is connected to the cuff cavity through the opening 11.

At least two marks 37, 38 (FIG. 7) are made on the surface of the tube body 2, the first mark 37 is located in the immediate vicinity of the cuff, the second mark is located at a distance of about 2-2.5 cm from the first mark, closer to the proximal end 3 of the tube, so that said second mark 38 is seen from the laryngopharynx when the endotracheal tube is in the required working position in the patient's larynx at the border of the upper part of the larynx.

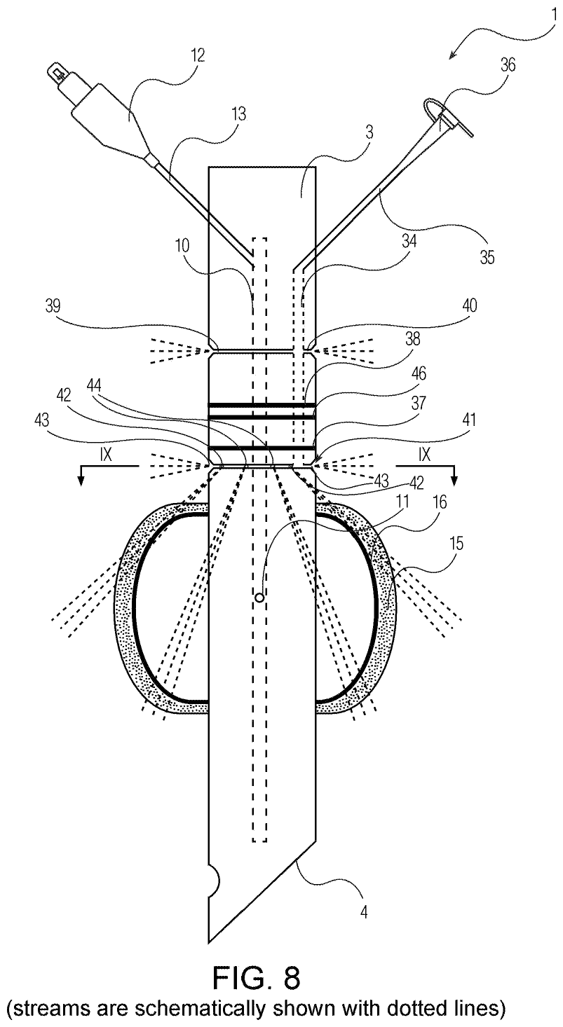

The endotracheal tube 1 (FIG. 8) comprises two channels 39, 40 that are similar to channels 24 and 25 of the second embodiment. Channels 39 and 40 are made in the tubular body 2 in a plane perpendicular to the axis of the tube 2, the channels 39, 40 are opened to the outside of the tube in the immediate vicinity of the second mark 38 from the side of the proximal end 3 of the tube. Said two channels 39, 40 communicate with the longitudinal drag supply channel 34 and having each a cone-shaped expanding outlet.

According to the third embodiment the endotracheal tube 1 (FIG. 8) comprises an annular drag supply channel 41 for supplying a drag to the area of contact of the cuff with the tracheal mucosa and to underlying parts of the respiratory tract. The annular drag supply channel 41 is made in the tube body 2 in the immediate vicinity of the inflatable cuff from the proximal end 4 of the tube, between the cuff and the first mark 37.

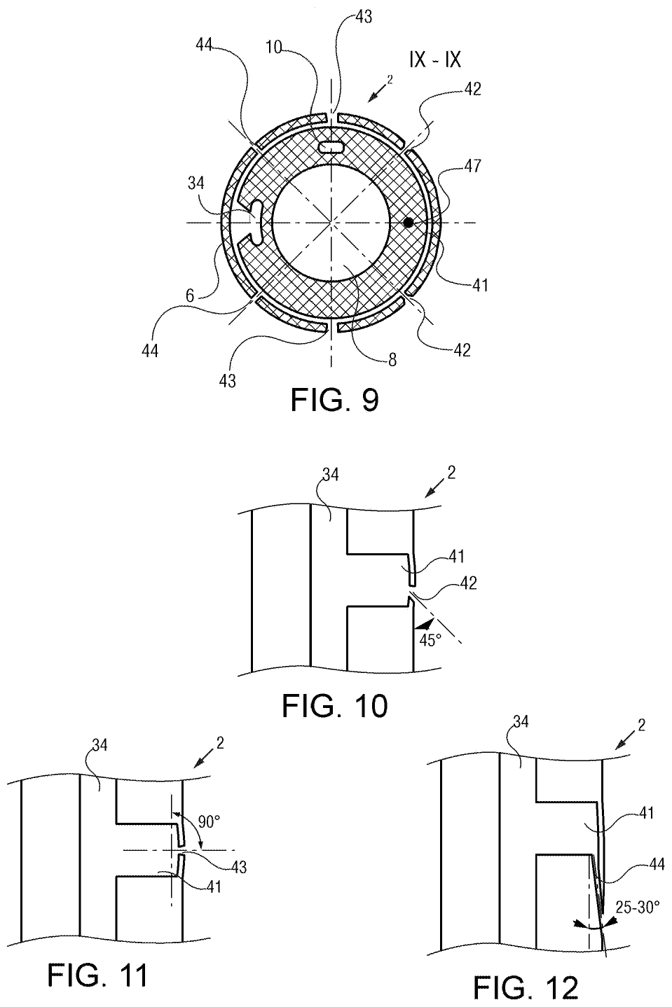

The annular drag supply channel 41 (FIG. 9) communicates with the longitudinal drag supply channel 34 and comprises: at least three pairs of holes 42, 43, 44 including the first pair, the second pair and the third pair, the holes 42, 43, 44 have axes of different directions. The holes 42, 43, 44 are located along the circumference at a distance from each other. The holes 42 of the first two pairs have a cone-shaped expanding shape with a large cross-sectional diameter closer to the outer surface of the wall of the tube, the axis of each hole 42 of the first pair is located at an angle of 45 degrees to the axis of the tube body 2 and is directed towards the distal end of the tube. The axis of each hole 43 of the second pair is at an angle of 90 degrees to the axis of the tube body 2. The holes 43 of the third pair have a cylindrical shape and the axes of the holes 43 of the third pair are located at an angle of 25-30 degrees to the longitudinal axis of the tube body 2 and are directed towards the distal end of the tube.

In the described embodiment, the inflatable cuff 45 is similar to the cuff 9 of the first embodiment of the endotracheal tube.

The endotracheal tube in the third embodiment comprises an additional mark 46 (FIG. 8) located in the immediate vicinity of the second mark 38 on the side of the first mark 37 at a distance of about 0.5 cm from the mark 38.

The pairs of holes 42, 43, 44 of different directions are preferably made at a distance from each other along the circumference on the concave side 5 of the tube body 2.

FIG. 9 shows X-ray mark 47 passing inside the wall of the tube body 2 along the entire length, said mark 47 in some cases is necessary for X-ray control of the depth of immersion of the tube body 2 into the trachea during surgery.

In all three embodiments of the endotracheal tube 1, it is preferable that the outer porous layer 15 of biocompatible polymeric material is thickened in the area located in the middle part of the inflatable cuff 9, in the zone of contact of the cuff with the trachea.

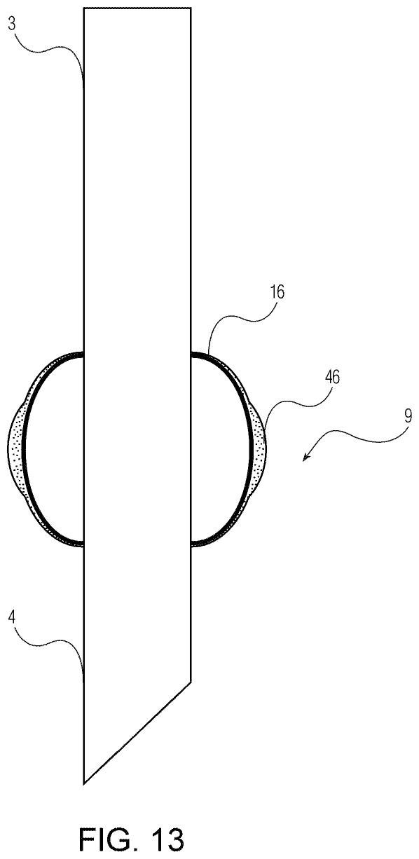

FIG. 13 schematically shows the location of the inflatable cuff 9 near the distal end 4 of the tube body 2, an embodiment in which the outer porous layer 15 of biocompatible polymeric material is thickened in the area located in the middle part of the inflatable cuff, in the contact zone of the cuff with the trachea.

The operation of the proposed endotracheal tube 1 is as follows.

In each of the three embodiments, before using the endotracheal tube 1, the condition of the inflatable cuff 9 (FIG. 2) is checked, in particular, its tightness.

At the beginning of intubation, the cuff 9 is deflated, air is pumped into the cuff after the endotracheal tube is inserted into the trachea.

The protective film 17 is removed from the cuff 9. The film 17 protects the outer porous layer 15, comprising a uniformly distributed local anesthetic of a single dose of anesthetic for adults or children, respectively. After that, the endotracheal tube 1 is inserted into the larynx.

According to the first embodiment, the endotracheal tube 1 is installed so that the patient's glottis is between the two marks 14, 18 (FIG. 2).

During intubation, when the cuff is not inflated, passive contact of the layer 15 drug with the tracheal mucosa occurs. After the installation of the tube 1, the cuff 9 is inflated, while the drug is released actively and uniformly in the amount of a single dose from the porous outer layer 15 of the cuff and the drag impacts on the tracheal mucosa.

If necessary, at the end of the action of the local anesthetic, the drug is injected in the form of a solution through the catheter. For this, the endotracheal tube of the second or third embodiment is used.

According to the second embodiment, the endotracheal tube 1 (FIG. 5) is set in the working position so that the glottis is between the two marks 22 and 23.

After the endotracheal tube 1 is installed, the cuff is inflated, while the drug is released actively and uniformly in the amount of a single dose from the porous outer layer 15 of the cuff and the drag impacts on the tracheal mucosa.

When a clinical situation arises that requires the introduction of a local anesthetic, a drug is injected into the irrigation port 20, the drug is supplied through channels 30 and 31 and provides anesthesia of the tracheal mucosa closer to the distal end of the tube, as well as through channels 28 and 29 to the tracheal mucosa from the proximal end, and through channels 24 and 25 into the region of the space under ligaments with under-ligaments structures. After that, the doctor moves the endotracheal tube 2 cm below in the patient, and re-delivers the drug through channels 28 and 29, providing anesthesia of the tracheal mucosa, and anesthesia of the subglottic space is performed through channels 24 and 25, followed by placing the endotracheal tube in its original position.

According to the third embodiment of the endotracheal tube 1 (FIG. 7), after the protective film 17 is removed from the cuff, the doctor performs tracheal intubation, during which, under visual control, the doctor performs entering the estimated amount of the drug through the irrigation port 35 when the mark 37 passes behind the glottis. The drug is delivered through the holes 42-44 in the annular channel 41, in front of the distal end 4 of the tube, which leads to pre-anesthesia of the tracheal mucosa during intubation.

The endotracheal tube 1 (FIG. 7) is installed into the working position, wherein the glottis is located between the mark 38 and the additional mark 46.

After the endotracheal tube 1 is installed, the cuff is inflated, while the drug is released actively and uniformly in the amount of a single dose from the porous outer layer 15 of the cuff and the drag impacts on the tracheal mucosa.

When a clinical situation arises that requires the introduction of a local anesthetic, a drug is injected into the irrigation port 35, is fed through the holes 42-44 in the annular channel 41 into the contact zone of the cuff with the tracheal mucosa at the same time; the cuff is preliminarily deflated for the duration of the manipulation.

The drug is delivered to the subglottic space through holes 43 (FIG. 8), the axes of which are located at an angle of 90 degrees to the tube axis. The drug is supplied to the contact zone of the cuff with the tracheal mucosa through the holes 42, the axes of which are located at an angle of 45 degrees to the axis of the tube. The drug is delivered to the area of contact of the cuff with the tracheal mucosa through holes 44, the axes of which are located at an angle of 25-30 degrees to the axis of the tube, and further towards the distal end of the tube.

At the same time, the drug is supplied to the space under ligaments with under-ligaments structures for their anesthesia through channels 39 and 40.

After the end of the manipulation, the cuff is inflated again.

The doctor visualizes the position of the endotracheal tube by the position of the marks 37, 38, 46.

Thus, during tracheal intubation, safe and effective anesthesia of the mucous of the trachea and laryngopharynx occurs, which makes it possible to level the reflex hemodynamic response.

As a consequence, a decrease in the drug load is achieved, i.e. reducing the dose of general drugs and the dose of muscle relaxants, which helps to reduce the intoxication of the patient and a more successful course of the postoperative period.

### INDUSTRIAL APPLICABILITY

Endotracheal tubes are used in anesthesiology, intensive care, critical care medicine to manipulate the tracheobronchial tree, to administer various drugs. Endotracheal tubes can also be used to protect and maintain the patency of the upper airways when transporting the injured and injured during the stages of medical evacuation.

### EXAMPLES

### Example 1

Patient A., 55 years old. Diagnosis: Graves' disease, mild thyrotoxicosis, sub compensated.

Estimated anesthesia time: 60-90 minutes

Surgical treatment is planned: Thyroidectomy. Concomitant pathology: hypertension of the 2nd degree, allergic history without features. The general condition according to the results of the examination is satisfactory, there is no peripheral edema, vesicular breathing, no wheezing, clear heart sounds, rhythmic, heart rate—88 beats/min., blood pressure—140/90 mm Hg. Endotracheal anesthesia is planned. ASA (American Association of Anesthesiologists) risk—II degree. BIS monitoring was carried out at all stages of anesthesia.

Premedication: Fentanyl 0.005%-0.5 intravenously. Monoinduction in anesthesia with sevoflurane: insufflation of a gas-narcotic mixture at a set rate on the evaporator of 8% and a flow of 100% oxygen of 81/min. The cuff of the endotracheal tube is pre-checked for tightness, the protective film is removed from its porous layer containing the local anesthetic.

When using the endotracheal tube of the first embodiment, there is no need to calculate the amount of the drug to be introduced into the contact area of the cuff and the tracheal mucosa, since the entire drug is deposited in the porous layer 15 of the cuff 9 of the endotracheal tube. After 3.5 minutes, the achieved level of anesthesia (MAC 1.5 minimum alveolar concentration) made it possible to perform tracheal intubation with an endotracheal tube, without additional administration of analgesics and muscle relaxants. BIS monitoring indicators at this stage were ≥50.

Respiratory support: apparatus Fabius Plus XL anesthesia and respiratory.

Basic general anesthesia: assisted mechanical ventilation (IVL) in

Pressure Support mode with a maximum trigger sensitivity of 2 L/min, continuous inhalation of sevoflurane 1.5 MAC, continuous inhalation of sevoflurane 1.5 MAC and continuous infusion of fentanyl at an average rate of 1.0-1.5 mcg/kg/hour.

The working conditions of the surgeons were assessed as comfortable, stable hemodynamic parameters were noted (BP 130/70-140/70 mm Hg. Pulse 62-76 beats/min.) general anesthesia and analgesia were not required. Smooth awakening, tracheal extubation with stable hemodynamic parameters, observation in the operating room and transfer of the patient to the post-anesthesia ward of the specialized department were noted. Anesthesia time: 68 minutes.

### Example 2

Patient B., 68 years old. Diagnosis: Generalized atherosclerosis. Atherosclerosis of the extracranial branches of the BCA. ICA stenosis on the right up to 84%, ICA on the left up to 64%. SMN 4 tbsp.

Estimated anesthesia time: 90-120 minutes

Surgery planned: Carotid endarterectomy of the ICA on the right. Initial hemodynamic parameters: heart rate—72 beats/min., Blood pressure—160/70 mm Hg. Art. Endotracheal anesthesia is planned. ASA risk—II degree.

Premedication: Fentanyl 0.005%-0.5 intravenously. Monoinduction in anesthesia with sevoflurane: insufflation of a gas-narcotic mixture at a set rate on the evaporator of 8% and a flow of 100% oxygen of 81/min. After 3.5 minutes, the achieved level of anesthesia (MAC 1.5) made it possible to perform tracheal intubation with an endotracheal tube 1 of the second embodiment with an irrigation port, without additional administration of analgesics and muscle relaxants. Respiratory support: Dameca anesthesia and respiratory apparatus.

Basic general anesthesia: assisted mechanical ventilation (ALV) in Pressure Support mode with a maximum trigger sensitivity of 2 L/min, continuous inhalation of sevoflurane 1.5 MAC and continuous infusion of fentanyl at an average rate of 1.0-1.5 μg/kg/hour. Psupp 8 mm. water column.

In this clinical case, taking into account the expected duration of anesthesia (90-120 minutes), an endotracheal tube 1 is used, which has an additional irrigation port 19 for intraoperative administration of drugs. Before the operation, 80 mg of 2% lidocaine solution was injected into the irrigation port of the endotracheal tube. 62 minutes after the start of the operation, the patient showed elements of laryngeal-pharyngeal reflexes to the presence of an endotracheal tube and some tendency to systemic hypertension (BP 160/60-170/80 mm Hg. Pulse 88-100 beats/min).

A decision was made to inject additional local anesthetic (lidocaine 2% -80 mg) into the irrigation port. In the proposed design of the endotracheal tube, there is no need to deflate the cuff to supply the drug to the contact zone between the cuff and the tracheal mucosa, since when the cuff is deflated, the trachea-cuff contour is depressurized.

If it is necessary to supply the drug, the drug is injected through the holes 30, 31, after which the doctor moves the endotracheal tube 2 cm deeper and re-delivers the drug, followed by the installation of the endotracheal tube in its original position, without depressurizing the cuff circuit-trachea. In 5-7 minutes after administration, hemodynamic parameters stabilization (BP 135/70 mm Hg. Pulse 72-82 beats/min), leveling of laryngeal-pharyngeal reflexes to comfortable surgical conditions.

Thereafter, within 45 minutes, a smooth course of anesthesia, stable hemodynamics, awakening, extubation of the trachea were unremarkable. Observation in the operating room, transfer to the post-anesthesia ward of the specialized department.

Anesthesia time: 100 minutes.

### Example 3

Patient G., 62 years old. Diagnosis: thyroid cancer.

Estimated anesthesia time: more than 120 minutes

Surgical treatment is planned: Thyroidectomy with lymphodesection. Concomitant pathology: hypertension of the 3rd degree, the patient receives complex antihypertensive therapy, the treating therapist regularly changes the treatment regimen, but the course of the disease is quite resistant, which is confirmed by the performed Holter daily monitoring. The council made a decision to carry out the operation, taking into account the severity of the underlying disease and the need to start radiation therapy as soon as possible, the allergic history was unremarkable.

The general condition according to the results of the examination is satisfactory, there is no peripheral edema, vesicular breathing, no wheezing, clear heart sounds, rhythmic, heart rate—62 beats/min., Blood pressure—150/80 mm Hg. Art. Endotracheal anesthesia is planned. ASA risk—II degree. BIS monitoring was carried out at all stages of anesthesia.

Premedication: Fentanyl 0.005%-0.5 intravenously. Monoinduction in anesthesia with sevoflurane: insufflation of a gas-narcotic mixture at a set rate on the evaporator of 8% and a flow of 100% oxygen of 81/min.

Given the severe concomitant background (hypertension resistant to complex therapy), it is necessary to suppress the negative hemodynamic response to tracheal intubation as much as possible.

The proposed constructive solution of the endotracheal tube (third embodiment) first of all allows to ensure the supply of drugs in front of the distal end of the tube and conduct preliminary anesthesia of the tracheal mucosa during intubation, during which the nurse, at the command of the anesthesiologist, when visualizing the correct final location of the endotracheal tube, can enter the calculated amount of local anesthetic, excluding its ingress into the glottis.

In this clinical case, after 3.5 minutes, the achieved level of anesthesia (MAC 1.5) made it possible to perform tracheal intubation with an endotracheal tube with the introduction of 80 mg of lidocaine in this way, without additional administration of analgesics and muscle relaxants. Respiratory support: Mindrey anesthesia and breathing apparatus.

Basic general anesthesia: assisted mechanical ventilation (ALV) in Pressure Support mode with a maximum trigger sensitivity of 2 L/min, continuous inhalation of sevoflurane 1.5 MAC and continuous infusion of fentanyl at an average rate of 1.0-1.5 μg/kg/hour. Psupp 8 mm. water column. In this clinical case, taking into account the expected duration of anesthesia (more than 120 minutes), an endotracheal tube is used, which has an additional irrigation port 34 for intraoperative administration of drugs. Before the operation, 80 mg of 2% lidocaine solution was injected into the irrigation port of the endotracheal tube.

94 minutes after the start of the operation, the patient had elements of laryngeal-pharyngeal reflexes for the presence of an endotracheal tube and some tendency to systemic hypertension (BP 160/70-170/90 mm Hg. Pulse 82-96 beats/min).

A decision was made to inject additional local anesthetic (lidocaine 2% -80 mg) into the irrigation port. In 4 minutes after administration, stabilization of hemodynamic parameters (BP 142/80 mm Hg. Pulse 68-74 beats/min.) And leveling of laryngeal-pharyngeal reflexes were ensured, the working conditions of surgeons were assessed as comfortable, the entire main period of the operation remained independent. breathing, deepening of general anesthesia and analgesia was not required. Smooth awakening, tracheal extubation with stable hemodynamic parameters, observation in the operating room and transfer of the patient to the post-anesthesia ward of the specialized department were noted.

Anesthesia time: 145 minutes.

1. An endotracheal tube comprising:
a tubular body having a form of a flexible curved transparent tube with open proximal and distal ends,
a connector mounted on the proximal end of the tube and served for attaching a tube cavity to a ventilation device,
an inflatable cuff having a cavity, said inflatable cuff fixed hermetically around the distal end of the curved tube and configured to be inflated to seal the cuff against a wall of a patient's trachea and to hold the distal end of the endotracheal tube in the desired position in a patient's larynx,
a longitudinal air supply channel in the wall of the tubular body, said channel being in communication with the cavity of the inflatable cuff,
a sealed balloon having elastic walls and configured to maintain and monitor pressure in the cuff cavity, the sealed balloon connected to an air supply port, and connected via a catheter to the longitudinal air supply channel to supply an air into the cuff cavity, and
a mark made on the surface of the tube from the side of the proximal end at a distance of 2.5-3 cm from the inflatable cuff,
characterized in that the inflatable cuff comprises two layers, wherein the outer layer of the inflatable cuff is made of a porous biocompatible polymer material and serves as a depot for input and hold the drug in the pores of the polymer material and remove the drug from the pores of the zone of contacting of the cuff with the trachea when the cuff is inflated providing infusion of the drug into the tracheal mucosa.

2. An endotracheal tube according to claim 1, in which the outer porous layer of biocompatible polymeric material is thickened in the area located in the middle of the inflatable cuff, in the zone of contact of the cuff with the trachea.

3. The endotracheal tube according to claim 1, in which the inner layer of the inflatable cuff is made of homogeneous polyvinyl chloride, ensuring to inflate the cuff to seal the cuff against a wall of a patient's trachea to hold it in the trachea.

4. The endotracheal tube according to claim 1, in which the outer layer of the cuff is made of porous polyvinyl chloride.

5. The endotracheal tube according to claim 1, in which a local anesthetic is used as the drug.

6. The endotracheal tube according to claim 1, in which the drug is a form of a solution, suspension or ointment held in the pores of the porous material of the inflatable cuff.

7. The endotracheal tube according to claim 1, in which a single dose drug is used as an amount of the drug held in the pores of the porous material of the outer layer.

8. The endotracheal tube according to claim 1, comprising an additional mark located in the immediate vicinity of the first mark from the distal end of the endotracheal tube at a distance of about 0.5 cm from the first mark.

9. An endotracheal tube comprising:
a tubular body having a form of a flexible curved transparent tube with open proximal and distal ends,
a connector mounted on the proximal end of the tube and served for attaching a tube cavity to a ventilation device,
an inflatable cuff having a cavity, said inflatable cuff fixed hermetically around the distal end of the curved tube and configured to be inflated to seal the cuff against a wall of a patient's trachea and to hold the distal end of the endotracheal tube in the desired position in a patient's larynx,
a longitudinal air supply channel in the wall of the tubular body, said channel being in communication with the cavity of the inflatable cuff,
a sealed balloon having elastic walls and configured to maintain and monitor pressure in the cuff cavity, the sealed balloon connected to an air supply port, and connected via a catheter to the longitudinal air supply channel to supply an air into the cuff cavity,
a longitudinal drag supply channel, the channel made in the wall of the tubular body and connected by means of a catheter to a metered-dose drug supply container, wherein the central angle between the longitudinal air supply channel and the longitudinal drag supply channel is in the range of 90-180 degrees,
at least two marks made on the surface of the tube, the first mark is located in the immediate vicinity of the cuff, the second mark is located at a distance of about 2-2.5 cm from the first mark, closer to the proximal end of the tube, so that said second mark is seen from the laryngopharynx when the endotracheal tube is in the required working position of the endotracheal tube in the patient's larynx at the border of the upper part of the larynx,
two channels in the tubular body in a plane perpendicular to the axis of the tube, the channels opened to the outside of the tube in the immediate vicinity of the second mark from the side of the proximal end of the tube, said two channels communicating with the longitudinal drag supply channel and having each a cone-shaped expanding outlet,
two channels in the tubular body in a plane perpendicular to the axis of the tube, the channels opened to the outside of the tube in the immediate vicinity of the cuff between said first mark and the cuff, said two channels communicating with the longitudinal drag supply channel and having each a cone-shaped expanding outlet, and
two channels in the tubular body in a plane perpendicular to the axis of the tube, the channels opened to the outside of the tube in the immediate vicinity of the distal end of the tube between the cuff and the distal end of the tube, said two channels communicating with the longitudinal drag supply channel and having each a cone-shaped expanding outlet,
characterized in that the inflatable cuff comprises two layers, wherein the outer layer of the inflatable cuff is made of a porous biocompatible polymer material and serves as a depot for input and hold the drug in the pores of the polymer material and remove the drug from the pores of the zone of contacting of the cuff with the trachea when the cuff is inflated providing infusion of the drug into the tracheal mucosa.

10. An endotracheal tube according to claim 9, in which the outer porous layer of biocompatible polymeric material is thickened in the area located in the middle of the inflatable cuff, in the zone of contact of the cuff with the trachea.

11. The endotracheal tube according to claim 9, in which the inner layer of the inflatable cuff is made of homogeneous polyvinyl chloride, ensuring to inflate the cuff to seal the cuff against a wall of a patient's trachea to hold it in the trachea.

12. The endotracheal tube according to claim 9, in which the outer layer of the cuff is made of porous polyvinyl chloride.

13. The endotracheal tube according to claim 9, in which a local anesthetic is used as the drug.

14. The endotracheal tube according to claim 9, in which the drug is a form of a solution, suspension or ointment held in the pores of the porous material of the outer layer.

15. The endotracheal tube according to claim 9, in which a single dose drug is used as an amount of the drug held in the pores of the porous material of the inflatable cuff.

16. The endotracheal tube according to claim 9, comprising an additional mark located in the immediate vicinity of the first mark from the distal end of the endotracheal tube at a distance of about 0.5 cm from the first mark.

17. An endotracheal tube comprising:
a tubular body having a form of a flexible curved transparent tube with open proximal and distal ends,
a connector mounted on the proximal end of the tube and served for attaching a tube cavity to a ventilation device,
an inflatable cuff having a cavity, said inflatable cuff fixed hermetically around the distal end of the curved tube and configured to be inflated to seal the cuff against a wall of a patient's trachea and to hold the distal end of the endotracheal tube in the desired position in a patient's larynx,
a longitudinal air supply channel in the wall of the tubular body, said channel being in communication with the cavity of the inflatable cuff,
a sealed balloon having elastic walls and configured to maintain and monitor pressure in the cuff cavity, the sealed balloon connected to an air supply port, and connected via a catheter to the longitudinal air supply channel to supply an air into the cuff cavity,
a longitudinal drag supply channel, the channel made in the wall of the tubular body and connected by means of a catheter to a metered-dose drug supply container, wherein the central angle between the longitudinal air supply channel and the longitudinal drag supply channel is in the range of 90-180 degrees,
at least two marks made on the surface of the tube, the first mark is located in the immediate vicinity of the cuff, the second mark is located at a distance of about 2-2.5 cm from the first mark, closer to the proximal end of the tube, so that said second mark is seen from the laryngopharynx when the endotracheal tube is in the required working position of the endotracheal tube in the patient's larynx at the border of the upper part of the larynx,
two channels in the tubular body in a plane perpendicular to the axis of the tube, the channels opened to the outside of the tube in the immediate vicinity of the second mark from the side of the proximal end of the tube, said two channels communicating with the longitudinal drag supply channel and having each a cone-shaped expanding outlet,
an annular drag supply channel for supplying the drag to the area of contact of the cuff with the tracheal mucosa and to underlying parts of the respiratory tract, the annular drag supply channel made in the tube wall in the immediate vicinity of the inflatable cuff from the proximal end of the tube, between the cuff and the first mark, the annular drag supply channel communicating with the longitudinal drag supply channel and comprising:
at least three pairs of holes including the first pair, the second pair and the third pair, the holes having axes of different directions, the holes are located along the circumference at a distance from each other, wherein the first two pairs of holes having a cone-shaped expanding shape with a large cross-sectional diameter closer to the outer surface of the wall of the tube, the axis of each hole of the first pair is located at an angle of 45 degrees to the axis of the tube and is directed towards the distal end of the tube, the axis of each hole of the second pair is placed at an angle of 90 degrees to the axis of the tube, the third pair of holes has a cylindrical shape and the axes of the holes of the third pair are located at an angle of 25-30 degrees to the longitudinal axis of the tube and are directed towards the distal end of the tube,
characterized in that the inflatable cuff comprises two layers, wherein the outer layer of the inflatable cuff is made of a porous biocompatible polymer material and serves as a depot for input and hold the drug in the pores of the polymer material and remove the drug from the pores of the zone of contacting of the cuff with the trachea when the cuff is inflated providing infusion of the drug into the tracheal mucosa.

18. An endotracheal tube according to claim 17, in which the outer porous layer of biocompatible polymeric material is thickened in the area located in the middle of the inflatable cuff, in the zone of contact of the cuff with the trachea.

19. The endotracheal tube according to claim 17, in which a single dose drug is used as an amount of the drug held in the pores of the porous material of the outer layer.

20. The endotracheal tube according to claim 17, in which the inner layer of the inflatable cuff is made of homogeneous polyvinyl chloride, ensuring to inflate the cuff to seal the cuff against a wall of a patient's trachea to hold it in the trachea.

21. The endotracheal tube according to claim 17, in which the outer layer of the cuff is made of porous polyvinyl chloride.

22. The endotracheal tube according to claim 17, in which a local anesthetic is used as the drug.

23. The endotracheal tube according to claim 17, in which the drug is a form of a solution, suspension or ointment held in the pores of the porous material of the inflatable cuff.

24. The endotracheal tube according to claim 17, comprising an additional mark in the immediate vicinity of the second mark from the side of the first mark.
